(19) United States
(12) Patent Application Publication
(10) Pub. No.: US 2026/0108472 A1
(43) Pub. Date: Apr. 23, 2026

(54) REJUVENATING THE AGED SKELETAL MICROENVIRONMENT TO REVERSE AGE-RELATED BONE LOSS

(71) Applicant: THE BOARD OF REGENTS OF THE UNIVERSITY OF TEXAS SYSTEM, Austin, TX (US)

(72) Inventors: Xiao-Dong CHEN, San Antonio, TX (US); Milos MARINKOVIC, San Antonio, TX (US); Qiuxia DAI, San Antonio, TX (US)

(73) Assignee: THE BOARD OF REGENTS OF THE UNIVERSITY OF TEXAS SYSTEM, Austin, TX (US)

(21) Appl. No.: 19/341,099

(22) Filed: Sep. 26, 2025

Related U.S. Application Data
(60) Provisional application No. 63/699,636, filed on Sep. 26, 2024.

(51) Int. Cl. A61K 9/51; A61K 38/17; A61K 38/29; A61P 19/10

(52) U.S. Cl. CPC A61K 9/5115; A61K 38/1709; A61K 38/29; A61P 19/10

(57) ABSTRACT

Disclosed are methods and compositions for restoring Cyr61 in bone tissue. In certain aspects, the compositions comprise a recombinant Cyr61 carried in carbon nanotubes. The compositions may be used for various methods including restoring Cyr61 expression in bone, increasing bone density, or treating a bone disease in a patient.

### CROSS-REFERENCE TO RELATED APPLICATIONS

This application claims the benefit of priority to U.S. Provisional Patent Application Ser. No. 63/699,636, filed Sep. 26, 2024, which is hereby incorporated by reference in its entirety.

### FIELD OF INVENTION

The present invention concerns the fields of stem cell biology, bone biology, and regenerative medicine.

### BACKGROUND

Currently, treatments for age-related bone loss (osteoporosis) are limited, mainly focusing on decreasing bone resorption by reducing osteoclast activity (e.g., bisphosphonates and denosumab) or increasing bone formation by promoting osteoblast activity and preventing apoptosis (e.g., intermittent PTH and the estrogen mimetic, raloxifene). Nevertheless, these approaches have considerable side effects—the former has been shown to have more than a 50% chance of causing bisphosphonate-related osteonecrosis of the jaw (BRONJ), while the latter is not suitable for patients with cardiovascular or renal complications, and bone disorders such as Paget's disease.

Overall, while methods for restoring bone loss exist, the need for improvements in this field persists in light of at least the aforementioned drawbacks for the conventional methods.

### SUMMARY

The current inventors have identified a solution to the above-mentioned problems associated with bone loss. The solution resides in providing Cyr61, a matricellular protein lost during aging, carried by carbon nanotubes to a patient. This can be beneficial for at least restoring Cyr61 expression in the bone, increasing parathyroid hormone (PTH) receptor expression, increasing responsiveness to PTH therapies, increasing bone density, reversing bone loss, and treating osteopenia and/or osteoporosis. Therefore, the methods of the present disclosure provide a technical solution to at least some of the problems associated with the conventional methods for treating bone diseases.

Aspects of the disclosure include compositions comprising recombinant Cyr61 carried in carbon nanotubes. In some aspects, the carbon nanotubes comprise multi-walled carbon nanotubes. In some aspects, the carbon nanotubes are bone-targeting carbon nanotubes. In some aspects, the recombinant Cyr61 and carbon nanotubes are at approximately a 2:1 ratio. In some aspects, the recombinant Cyr61 and carbon nanotubes are at a 5:1, 4:1, 3:1, 2:1, 1:1, 1:2, 1:3, 1:4, 1:5 ratio, or any range derivable therein. In some aspects, the composition is formulated for intravenous administration. In some aspects, the recombinant Cyr61 is at a concentration of approximately 2 milligram per gram of composition. In some aspects, the recombinant Cyr61 is at a concentration of at least, at most, or approximately 0.5, 1, 2, 3, 4, 5 milligram per gram of composition, or any range derivable therein. In certain aspects, the recombinant Cyr61 is encapsulated in the carbon nanotubes.

In some aspects, the disclosure provides nanotube-based compositions configured to selectively accumulate in bone tissue. In some aspects, the bone-affinity ligands include bisphosphonates, tetracycline analogs, aspartate-rich peptides, osteocalcin-derived motifs, or catechol-containing moieties that bind hydroxyapatite. In some aspects, the nanotubes can be coated with calcium-binding polymers such as poly(aspartic acid) or poly(glutamic acid) to further enhance bone adherence. In some aspects, the compositions incorporate endosomal escape motifs to facilitate intracellular delivery to osteoclasts, osteoblasts, or osteocytes. In some aspects, the nanotubes exhibit an average outer diameter between about 2 nm and about 200 nm and a length between about 50 nm and about 10 μm. In some aspects, the nanotubes are single-walled, double-walled, or multi-walled structures with surface areas sufficient to load therapeutic or diagnostic payloads. In some aspects, the bone-targeting ligand is tethered to the nanotube via cleavable linkers responsive to acidic pH or enzymatic activity present in the bone resorption microenvironment.

Also disclosed are methods of targeting Cyr61 to bone tissue in a patient, methods of increasing bone marrow-derived mesenchymal stem cells, and methods for treating a patient. In some aspects, the method comprises one or more steps including any of: administering a composition comprising Cyr61 carried in carbon nanotubes to the patient, administering a PTH therapy to the patient, and monitoring the patient for bone density. In some aspects, the method comprises administering a composition comprising Cyr61 carried in carbon nanotubes to the patient. The composition can be any composition described herein. In some aspects, the patient has, is suspected of having, is diagnosed with having, or has symptoms of osteopenia or osteoporosis. The patient may have been determined to have reduced bone density. The patient may have been determined to have bone loss. In some aspects, the patient has been found to have reduced Cyr61, parathyroid hormone (PTH), and/or PTH receptor compared to a control. In some aspects, the control is a measured amount of Cyr61, PTH, and/or PTH in a healthy individual. In some aspects, the control is an average amount of Cyr61, PTH, and/or PTH in a healthy population. In some aspects, the control is a baseline amount of Cyr61, PTH, and/or PTH measured in the patient. In some aspects, the patient has been found to have reduced Cyr61, parathyroid hormone (PTH), and/or PTH receptor in a bone tissue sample from the patient. In some aspects, the patient is elderly. In some aspects, the composition is administered intravenously. In some aspects, the method comprises administering PTH and/or a PTH analog to the patient. In some aspects, the method comprises administering an intermittent PTH therapy and/or an intermittent PTH analog therapy to the patient.

Also disclosed are the following enumerated aspects:
Aspect 1 includes a composition comprising recombinant Cyr61 carried in carbon nanotubes.
Aspect 2 depends upon Aspect 1, wherein the carbon nanotubes comprise multi-walled carbon nanotubes.
Aspect 3 depends upon Aspect 1 or 2, wherein the carbon nanotubes are bone-targeting carbon nanotubes.
Aspect 4 depends upon any one of Aspects 1 to 3, wherein the recombinant Cyr61 and carbon nanotubes are at approximately a 2:1 ratio.
Aspect 5 depends upon any one of Aspects 1 to 4, wherein the composition is formulated for intravenous administration.
Aspect 6 depends upon any one of Aspects 1 to 5, wherein the recombinant Cyr61 is at a concentration of approximately 2 milligram per gram of composition.
Aspect 7 includes a method of targeting Cyr61 to bone tissue in a patient, the method comprising administering the composition of any one of Aspects 1 to 6 to the patient.
Aspect 8 depends upon Aspect 7, wherein the patient has been found to have reduced Cyr61, parathyroid hormone (PTH), and/or PTH receptor compared to a control.
Aspect 9 depends upon Aspect 8, wherein the control is a measured amount of Cyr61, PTH, and/or PTH in a healthy individual.
Aspect 10 depends upon Aspect 8, wherein the control is an average amount of Cyr61, PTH, and/or PTH in a healthy population.
Aspect 11 depends upon Aspect 8, wherein the control is a baseline amount of Cyr61, PTH, and/or PTH measured in the patient.
Aspect 12 depends upon any one of Aspects 7 to 11, wherein the patient has been found to have reduced Cyr61, parathyroid hormone (PTH), and/or PTH receptor in a bone tissue sample from the patient.
Aspect 13 depends upon any one of Aspects 7 to 12, wherein the patient is elderly.
Aspect 14 depends upon any one of Aspects 7 to 13, wherein the composition is administered intravenously.
Aspect 15 depends upon any one of Aspects 7 to 14, further comprising administering PTH and/or a PTH analog to the patient.
Aspect 16 depends upon any one of Aspects 7 to 14, further comprising administering an intermittent PTH therapy and/or an intermittent PTH analog therapy to the patient.
Aspect 17 includes a method of increasing bone marrow-derived mesenchymal stem cells, the method comprising administering the composition of any one of Aspects 1 to 6 to the patient.
Aspect 18 depends upon Aspect 17, wherein the patient has been found to have reduced Cyr61, parathyroid hormone (PTH), and/or PTH receptor compared to a control.
Aspect 19 depends upon Aspect 18, wherein the control is a measured amount of Cyr61, PTH, and/or PTH in a healthy individual.
Aspect 20 depends upon Aspect 18, wherein the control is an average amount of Cyr61, PTH, and/or PTH in a healthy population.
Aspect 21 depends upon Aspect 18, wherein the control is a baseline amount of Cyr61, PTH, and/or PTH measured in the patient.
Aspect 22 depends upon any one of Aspects 17 to 21, wherein the patient has been found to have reduced Cyr61, parathyroid hormone (PTH), and/or PTH receptor in a bone tissue sample from the patient.
Aspect 23 depends upon any one of Aspects 17 to 22, wherein the patient is elderly.
Aspect 24 depends upon any one of Aspects 17 to 23, wherein the composition is administered intravenously.
Aspect 25 depends upon any one of Aspects 17 to 24, further comprising administering PTH and/or a PTH analog to the patient.
Aspect 26 depends upon any one of Aspects 17 to 24, further comprising administering an intermittent PTH therapy and/or an intermittent PTH analog therapy to the patient.
Aspect 27 includes a method for treating a patient having, suspected of having, diagnosed with having, or having symptoms of osteopenia or osteoporosis, the method comprising administering the composition of any one of Aspects 1 to 6 to the patient.
Aspect 28 depends upon Aspect 27, wherein the patient has been found to have reduced Cyr61, parathyroid hormone (PTH), and/or PTH receptor compared to a control.
Aspect 29 depends upon Aspect 28, wherein the control is a measured amount of Cyr61, PTH, and/or PTH in a healthy individual.
Aspect 30 depends upon Aspect 28, wherein the control is an average amount of Cyr61, PTH, and/or PTH in a healthy population.
Aspect 31 depends upon Aspect 28, wherein the control is a baseline amount of Cyr61, PTH, and/or PTH measured in the patient.
Aspect 32 depends upon any one of Aspects 27 to 31, wherein the patient has been found to have reduced Cyr61, parathyroid hormone (PTH), and/or PTH receptor in a bone tissue sample from the patient.
Aspect 33 depends upon any one of Aspects 27 to 32, wherein the patient is elderly.
Aspect 34 depends upon any one of Aspects 27 to 33, wherein the composition is administered intravenously.
Aspect 35 depends upon any one of Aspects 27 to 34, further comprising administering PTH and/or a PTH analog to the patient.
Aspect 36 depends upon any one of Aspects 27 to 34, further comprising administering an intermittent PTH therapy and/or an intermittent PTH analog therapy to the patient.

The terms “about” or “approximately” are defined as being close to as understood by one of ordinary skill in the art. In one non-limiting aspect the terms are defined to be within 10%, preferably, within 5%, more preferably, within 1%, and most preferably, within 0.5%.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present disclosure can “comprise,” “consist essentially of,” or “consist of” particular ingredients, components, compositions, etc., disclosed throughout the specification. With respect to the phrase “consisting essentially of,” a basic and novel property of the compositions and methods of the present disclosure is the ability to restore stem cell properties.

Throughout this application, the term “aging” is used to indicated the sum of processes, by which stem cell populations decrease in quantity and/or quality.

Throughout this application, the term “young” refers to humans (male or female) age 25 years and under, and also refers to the cells obtained from them.

Throughout this application, the term “elderly”, “old”, or “older” refers to humans (male or female) age 65 years and older, and also refers to the cells obtained from them.

Throughout this application, the term “subject”, “patient”, or “donor” refers to a male or female human.

Other objects, features and advantages of the present disclosure will become apparent from the following figures, detailed description, and examples. It should be understood, however, that the figures, detailed description, and examples, while indicating specific aspects of the disclosure, are given by way of illustration only and are not meant to be limiting. Additionally, it is contemplated that changes and modifications within the spirit and scope of the disclosure will become apparent to those skilled in the art from this detailed description. In further aspects, features from specific aspects may be combined with features from other aspects. For example, features from one aspect may be combined with features from any of the other aspects. In further aspects, additional features may be added to the specific aspects described herein.

### DESCRIPTION OF THE DRAWINGS

For a more complete understanding, reference is now made to the following descriptions taken in conjunction with the accompanying drawings, in which:

FIGS. 1A-1D: (FIG. 1A) Wb analysis of Cyr61 in L1-5 vertebrae at 1, 2 and 4 weeks after treatment of old mice with rCyr61/MWCNTs; (FIG. 1B) Quantification of Cyr61 bands on Wb in (A), normalized to young mice; (FIG. 1C) Representative images of CFU-F and CFU-OB stained with crystal violet (blue) and von Kossa (dark), respectively; (FIG. 1D) Quantification of CFU-F and CFU-OB; 3 or 1×10⁶ freshly isolated BM cells were initially seeded into 6-well plates; the number of CFUs per 1×10⁶ BM cells was determined at the various time points.

FIGS. 2A-2E: (FIG. 2A) Wb analysis of Cyr61 in L1-3 vertebrae at 160 days after the first injection (given 3 times, separated by 2 weeks) of rCyr61/MWCNTs (C6/Pt) or PBS alone in 12-month-old mice (6 mice/treatment). Y: 3-month-old mouse (young control). (FIG. 2B) Representative images of CFU-F and CFU-OB stained with crystal violet (blue) and von Kossa (dark), respectively. CFUs were assayed in 3 or 1×10⁶ freshly isolated BM cells plated into 6-well plates. (FIG. 2C) Quantification of CFU-F and CFU-OB, representing the number of CFUs per 1×10⁶ BM cells. *P<0.05 (n=3), vs. PBS treated. (FIG. 2D) μCT images of the distal femoral trabecular bone from 3 individual mice. (FIG. 2E) Quantification of femoral trabecular BMD. *P<0.05 (n=3), vs. PBS treated.

FIG. 3: Structure of Cyr61 Domains. SP (N-terminal secretory signal peptide); IGF-binding domain (module I); VWC (module II) binding to TGFβ/BMP; TSP-1 (module III) binding to αᵥβ₃ and α₆β₁ integrins to form a complex with LRP-1 (lipoprotein receptor-related protein 1); and a cysteine knot domain (module IV) binding to α₆β₁, type I collagen, fibronectin, and HSPG.

FIGS. 4A-4H: (FIGS. 4A & 4B) Wb analysis of proteins extracted from L1-3; Cyr61 & PTHIR band intensity normalized to β-actin. *P<0.05 (n=8) vs treatment, respectively. (FIGS. 4C & 4D) CFUs generated by BM-MSCs; CFU-F (crystal violet stain) and CFU-OB (Von Kossa stain), *P<0.05 (n=8), vs. C6/Pt-treated. (FIG. 4E) μCT analysis of lumbar vertebrae (L5) cortical bone mass (BV/TV) *P<0.05 (n=4), vs. C6/Pt. (FIGS. 4F-4H) RT-PCR of the indicated osteoblast markers in RNA extracted from femoral bone after BM removal. *P<0.05 (n=4), vs. C6/Pt.

FIGS. 5A-5G: (FIGS. 5A & 5B) Wb analysis of proteins extracted from L1-3; Cyr61 & PTHIR band intensity normalized to β-actin. Y: young mouse (3-month-old). *P<0.05 (n=6) vs treatment, respectively. (FIGS. 5C & 5D) CFUs generated by BM-MSCs; CFU-F (crystal violet stain) and CFU-OB (Von Kossa stain), *P<0.05 (n=6), vs. C6/Pt-treated. (FIG. 5E) Top panel: μCT images of distal femur trabecular bone (n=3). Lower panel: Histological sections of the femoral epiphysis stained with H&E (n=3). (FIG. 5F) Trabecular BMD of the femur quantified by μCT, *P<0.05 (n=6), vs. C6/Pt treated. (FIG. 5G) Whole body BMD measured by DXA at the indicated times after the first injection of C6/Pt, normalized to the BMD of the same individual before the treatment. *P<0.05 (n=6), vs. PBS. Blue arrows indicate the time injections were given.

FIG. 6: Phase contrast & immunofluorescence (IF) images of bone from mice treated with rCyr61/MWCNTs (C6/Pt) or PBS. Femoral bone sections were stained for IF microscopy with antibodies against mouse Cyr61 (red) and nuclei stained with DAPI (blue). The phase contrast field of view is adjacent to that of the IF section. White arrows identify the periosteum, orange arrows identify the endosteum. BM: bone marrow; C: cortical bone.

FIGS. 7A-7D: (FIG. 7A) Confirmation that Cyr61 was depleted in Cyr61− HS-5 cells (n=6/group). (FIG. 7B) PTHIR expression, *P<0.05 (n=6), vs. Cyr61− cells on TCP (T) at the same dose; IP<0.05, vs. Cyr61− cells on TCP (T) at the same dose. (FIG. 7C) Runx2 expression, *P<0.05 (n=6), vs. Cyr61− cells on TCP (T) or ECM (E) at the same dose; [symbol] P<0.05, vs. Cyr61− cells on TCP (T) at the same dose. (FIG. 7D) Immunofluorescent staining of PTHIR (red) on the surface of Wt vs. Cyr61− HS-5 cells maintained on TCP or ECM and treated/untreated with BMP-2 (200ng/ml). Cell nuclei were stained with Dapi (blue). Cells grown on ECM were more densely and evenly distributed across the culture surface than cells on TCP. Scale bar: 100 μm.

### DETAILED DESCRIPTION

Certain aspects herein provide a new treatment paradigm by replenishing Cyr61 (a key ECM component) that is lost from the MSC niche during aging, potentially preventing or retarding age-related bone loss, and providing a more efficient, effective, and less toxic treatment for osteoporosis, without requiring daily injections (e.g., PTH). Certain aspects allow for a smaller dosage of the therapeutic agents due to the bone-targeting properties of the carbon nanotubes described herein. Certain aspects herein also allow for reducing the dosage of Cyr61 or other components by encapsulating the components in carbon nanotubes. In addition, certain aspects increase the anabolic effects of intermittent PTH, including by prior replenishment of Cyr61 to restore or at least improve the properties of the aging MSC microenvironment (niche).

These and other non-limiting aspects of the present disclosure are discussed in further detail in the following sections.

### A. The Extracellular Matrix

Besides its obvious roles in determining the architecture and mechanical properties of tissues, the ECM greatly influences cell adhesion, migration, proliferation, differentiation, and survival. ECM modulates the bioactivities of growth factors and cytokines, such as transforming growth factor-β (TGF-β), tumor necrosis factor-α, and platelet-derived growth factor, by activating latent growth factors via proteolytic processing, by sequestering growth factors and hindering them from binding to their receptors, or by directly affecting receptor activity. Cells residing in the ECM not only receive ECM cues but also influence ECM signaling by secreting ECM components and by producing enzymes that cause proteolytic modification of proteins and growth factors in the ECM. The end result is a “give and take” relationship between cells and the ECM that defines cell behavior.

Regardless of tissue types, the ECM consists of collagen fibers, laminin polymers, cell adhesion proteins such as fibronectin, high molecular-weight proteoglycans, various growth factors that often exist in a latent or masked form, and members of the small leucine-rich proteoglycan (SLRP) family, mainly biglycan (bgn) and decorin (dcn) (Clark and Keating, 1995; Hocking et al., 1998; Lee et al., 1999). As might be expected from such a complex composition, the structure of the ECM in most tissues is not well understood. However, based on the studies of kidney basal lamina and ECM of skin, it is generally accepted that the ECM structure is dictated by the interaction of collagen fibers with each other and with laminin, as well as high-molecular-weight proteoglycans, resulting in the formation of an interlocking mesh-like structure (Pollard and Earnshaw, 2002). SLRPs such as bgn and den are also associated with collagen fibers and also with fibronectin and growth factors in the ECM. SLRPs appear to be important for collagen fibrillogenesis, as well as growth factor localization.

The loss of stemness during growth of MSCs using current culture methods reflects the production of more differentiated progeny with diminished self-renewal capacity, rather than the production of identical daughter stem cells. The term “stemness” refers to the stem cell properties including self-renewal (proliferation) and multipotentiality (capacity for the differentiation into multiple cell lineages). Involvement of the ECM in the regulation of mesenchymal colony forming units (MCFUs) is further supported by evidence that deletion of the ECM components biglycan and decorin has a deleterious effect on responsiveness of marrow derived osteoblast progenitors to BMPs and TGF-β (Di Gregorio et al., 2001; Chen et al., 2004). At this stage, it is unknown how the ECM regulates the behavior of MCFUs. Earlier work has shown that the ECM modulates the activity of growth factors by controlling proteolytic activation of latent factors, as occurs in the case of TGF-β (Dallas et al. 2002). The ECM also interacts with cell surface receptors to prevent binding of the cognate ligand, as occurs in the case of the epidermal growth factor (EGF) receptor (Santra et al., 2002), and sequesters factors such as platelet-derived growth factor (PDGF) and BMPs (Suzawa et al., 1999; Nili et al., 2003). The ECM may also bind growth-promoting factors from the serum for optimal presentation to MSCs. Finally, the ECM may enhance the function of putative accessory cells that support MCFU replication.

### B. Nanoparticles

Some aspects disclosed herein concern compositions comprising a nanoparticle, including carbon nanotubes. In some aspects, the nanoparticle encapsulate a therapeutic agent, which can be any of the therapeutic agents disclosed herein, including a recombinant Cyr61. The therapeutic agent(s) formulated in the nanoparticles can have improved pharmacokinetic and/or pharmacodynamic properties. The therapeutic agent(s) formulated in the nanoparticles can be better tolerated by a patient. The therapeutic agent(s) formulated in the nanoparticles, in some aspects, are more effectively delivered to cells to affect their function, such as effecting transcriptional changes, than naked therapeutic agent(s). In some aspects, the composition confers water solubility to hydrophobic agents, to combinations of hydrophobic agents, and/or to combinations of hydrophobic and hydrophilic agents.

In some aspects, when formulated, the dry weight % of one or more therapeutic agents present in the nanoparticle compositions is equal to or at least about: 0.1%, 0.5%, 1%, 2.5%, 5%, 7.5%, 10%, 12.5%, 15%, 20%, 22.5%, 25%, 27.5%, 30%, 32.5%, 35%, 37.5%, 40%, 42.5%, 45%, 47.5%, 50%, 52.5%, 55%, 57.5%, 60%, or any ranger derivable therein. In some aspects, the therapeutic agents are provided in an aqueous composition. In some aspects, the wet weight % of one or more therapeutic agents present in the composition (with water included) is equal to or at least about: 0.5%, 1%, 2.5%, 5%, 7.5%, 10%, 12.5%, 15%, 20%, 22.5%, 25%, 27.5%, 30%, or any range derivable therein. In some aspects, the one or more therapeutic agents may be provided in the wet composition at a concentration of greater than or equal to about: 0.001 mg/mL, 0.005 mg/mL, 0.01 mg/mL, 0.05 mg/mL, 0.1 mg/mL, 0.5 mg/mL, 1 mg/mL, 5 mg/mL, 20 mg/mL, 30 mg/mL, 50 mg/mL, 100 mg/mL, or any range derivable therein.

In some aspects, the therapeutic agents, collectively or individually, are present in the aqueous nanoparticle composition at a concentration of less than or equal to about: 150 mg/mL, 100 mg/mL, 75 mg/mL, 50 mg/mL, 25 mg/mL, 20 mg/mL, 10 mg/mL, 5 mg/mL, 2.5 mg/mL, 2 mg/mL, 1.5 mg/mL, 1 mg/mL, 0.5 mg/mL, 0.1 mg/mL, 0.05 mg/mL, 0.01 mg/mL, or ranges including and/or spanning the aforementioned values. In some aspects, the one or more therapeutic agents, collectively or individually, are present in the aqueous composition at a concentration of greater than or equal to about: 150 mg/mL, 100 mg/mL, 75 mg/mL, 50 mg/mL, 25 mg/mL, 20 mg/mL, 10 mg/mL, 5 mg/mL, 2.5 mg/mL, 2 mg/mL, 1.5 mg/mL, 1 mg/mL, 0.5 mg/mL, 0.1 mg/mL, 0.05 mg/mL, 0.01 mg/mL, or ranges including and/or spanning the aforementioned values. In some aspects, the one or more therapeutic agents, collectively or individually, are present in the composition at a dry wt. % of equal to or at least about: 0.1%, 0.5%, 1%, 2.5%, 5%, 7.5%, 10%, 12.5%, 15%, 20%, 22.5%, 25%, 27.5%, 30%, 32.5%, 35%, 37.5%, 40%, 42.5%, 45%, 47.5%, 50%, 52.5%, 55%, 57.5%, 60%, or ranges including and/or spanning the aforementioned values. In some aspects, the one or more therapeutic agents, collectively or individually, are present in the composition at a wet wt. % of equal to or at least about: 0.1%, 0.5%, 1%, 2.5%, 5%, 7.5%, 10%, 12.5%, 15%, 20%, 22.5%, 25%, 27.5%, 30%, 32.5%, 35%, 37.5%, 40%, 42.5%, 45%, 47.5%, 50%, 52.5%, 55%, 57.5%, 60%, or ranges including and/or spanning the aforementioned values. In some aspects, as disclosed elsewhere herein, the composition is aqueous, while in others it has been dried into a powder (that is free of or substantially free of water). In some aspects, where the composition has been dried, it comprises a water content of less than or equal to 20%, 15%, 10%, 7.5%, 5%, 2.5%, 1%, or ranges including and/or spanning the aforementioned values.

In some aspects, as disclosed elsewhere herein, the composition is aqueous (e.g., contains water) while in other aspects, the composition is dry (lacks water or substantially lacks water). In some aspects, the composition has been dried (e.g., has been subjected to a process to remove most or substantially all water). In some aspects, the composition comprises nanoparticles in water (e.g., as a solution, suspension, or emulsion). In other aspects, the composition is provided as a powder (e.g., that may be constituted or reconstituted in water). In some aspects, as disclosed elsewhere herein, the water content (in wt. %) of the composition is less than or equal to about: 30%, 20%, 10%, 5%, 2.5%, 1%, 0.5%, 0.1%, 0%, or ranges including and/or spanning the aforementioned values. In some aspects, as disclosed elsewhere herein, the water content (in wt. %) of the composition is greater than or equal to about: 50%, 60%, 70%, 80%, 85%, 90%, 92.5%, 95%, 97.5%, or ranges including and/or spanning the aforementioned values. In some aspects, the water is nanopure, deionized, USP grade, WFI, and/or combinations of the foregoing. In some aspects, the composition is a dried composition comprising a nanoparticle having weight ratios of a first therapeutic agent: a nanoscale coordination polymer (NCP): optionally a lipid source, and optionally a surfactant of 1 to 50:1 to 50:1 to 50:0 to 17.5.

In some aspects, as disclosed elsewhere herein, the nanoparticle composition provides particles in the nano-measurement range. In some aspects, the nanoparticle is spherical or substantially spherical. In some aspects, the nanoparticle is a carbon nanotube. Carbon nanotubes include nanoparticles that have cylindrical tubular structures with a diameter of nanometers and are formed by rolling graphene sheets The carbon nanotube can have diameters around 0.5-2 nanometers, including any range derivable therein. In certain aspects, the carbon nanotubes are multi-walled carbon nanotubes, which can consist or comprise nested single-wall carbon nanotubes. In some aspects, the size of the particle is measured by dynamic light scattering. In some aspects, the size of the particle is measured using a zeta-sizer. In some aspects, the size of the particle can be measured using Scanning Electron Microscopy (SEM). In some aspects, the size of the particle is measured using a cyrogenic SEM (cryo-SEM). Where the size of a nanoparticle is disclosed elsewhere herein, any one or more of these instruments or methods may be used to measure such sizes.

### C. Administration of Therapeutic Compositions

Aspects of the present disclosure are directed to treatment or prevention of one or more diseases or conditions, including any of the bone diseases or disorders described herein. In some aspects, the present disclosure relates to treatment or prevention of a disease or condition affected by Cyr61 and/or PTH. In some aspects, the present disclosure related to treatment or prevention of osteopenia and/or osteoporosis.

In some aspects, a nanoparticle composition comprising a recombinant Cyr61 is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

In some aspects, as disclosed elsewhere herein, the nanoparticle composition aids in absorption, bioavailability, or other pharmacokinetic properties of the active compound when administered to an individual, including by orally ingestion or intravenous administration. As disclosed elsewhere herein, some aspects pertain to the use of the nanoparticle based nanodelivery system to protect the active compound from degradation and/or precipitation in a solution comprising the active compound (e.g., in an aqueous composition for administration to a subject). In some aspects, use of the delivery systems, including the nanoparticles, disclosed herein result in improved bioavailability and/or absorption rate. For instance, in some aspects, the Cmax of an active compound is increased using a disclosed aspect, the Tmax of an active compound is decreased using an aspect as disclosed herein, and/or the AUC of an active compound is increased using a disclosed aspect.

In some aspects, a therapeutic agent (e.g., a recombinant Cyr61) is administered in a nanoparticle composition at a dose of between 0.1 mg/kg and 5000 mg/kg. In some aspects, the therapeutic agent is administered in a nanoparticle composition at a dose of at least, at most, or about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range or value derivable therein.

The quantity to be administered, both according to number of treatments and unit dose, depends on the treatment effect desired. An effective dose is understood to refer to an amount necessary to achieve a particular effect. It is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, 200, 300, 400, 500, or 1000 μg/kg, mg/kg, μg/day, or mg/day or any range derivable therein. Furthermore, such doses can be administered at multiple times during a day, and/or on multiple days, weeks, or months.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Upon formulation, solutions will be administered in a manner compatible with the dosage formulation and in such amount as is therapeutically or prophylactically effective. The formulations are easily administered in a variety of dosage forms, such as the type of injectable solutions described above.

### D. Method of Restoring Stem Cell Properties

Autologous stem cells are preferential for stem cell based treatments over allogeneic (HLA matched donor) stem cells. However, the stem cells from the individual in need of these stem cell based treatments often exhibit reduced or diminished stem cells properties, resulting in challenges to obtain fully healthy autologous stem cells. The method disclosed herein is capable of facilitating production of autologous stem cell cultures using stem cells having reduced or diminished stem cell properties.

Aspects of the disclosure include a method for restoring stem cell properties to stem cells in need thereof. In some aspects, the stem cells in need of restoring stem cell properties can include stem cells with decreased quality of stem cells. In some instances, the stem cells in need of restoring stem cell properties can include aged stem cells that have reduced or substantially no stem cell properties compared to young stem cells. In some aspects, the stem cells in need of restoring stem cell properties may include stem cells from an individual over an age of 55 years. In some aspects, the stem cells in need of restoring stem cell properties may include stem cells from an individual at an age of 55 to over 96 years old. Non-limiting examples of the stem cells include mesenchymal stem cells derived from various tissues including bone marrow, adipose tissue, cartilage tissue, or any combination thereof. In some aspects, the mesenchymal stem cells may include bone marrow mesenchymal stem cells, adipose mesenchymal stem cells, umbilical cord mesenchymal stem cells, or any combinations thereof.

In some aspects, the properties of stem cells may include capability for differentiation, capability for self-renewal, viability or any combination thereof. In some instances, the stem cells in need of restoring stem cell properties may have substantially no response to an differentiation inducer. In some instances, the differentiation inducer may include an osteoblastogenesis inducer, an adipogenesis inducer, a chondroblastogenesis inducer, a musclegenesis inducer, or any combination thereof. Non-limiting examples of the osteoblastogenesis inducer may include BMP-2. In some instances, the stem cells in need of restoring stem cell properties may have substantially no response to an adipogenesis inducer. Non-limiting examples of adipogenesis inducer may include rosiglitazone

In some instances, the response of stem cells in need of restoring stem cell properties to an osteoblastogenesis inducer (e.g., BMP-2) can be determined by treating the stem cells with 10 to 100 ng/ml of the osteoblastogenesis inducer for 1 to 7 days and testing Runx2 expression levels after the treatment of the osteoblastogenesis. In some aspects, there is substantially no increase in Runx2 expression level of the osteoblastogenesis inducer treated stem cells in need of restoring stem cell properties, as compared to Runx2 expression level for stem cells that are from the same donor but not treated with osteoblastogenesis inducer, thereby indicating substantially no response to the osteoblastogenesis inducer.

In some instances, the response of stem cells in need of restoring stem cell properties to an adipogenesis inducer (e.g., rosiglitazone) can be determined by treating the stem cells with about 1.9 μg/ml of the adipogenesis inducer for about 2 days and testing peroxisome proliferator-activated receptor gamma (PPARγ) expression levels after the treatment of adipogenesis. In some aspects, there is substantially no increase in PPARγ expression level of the adipogenesis inducer treated stem cells in need of restoring stem cell properties, as compared to PPARγ expression level for stem cells that are from the same donor but not treated with the adipogenesis inducer, thereby indicating substantially no response to the adipogenesis inducer.

In aspects of the disclosure, the method may include providing stem cells in need of restoring stem cell properties. In some aspects, the stem cells in need of restoring stem cell properties produce extracellular matrix that is deficient in CCN1/Cyr61 protein. In some instances, the stem cells in need of restoring properties is obtained from bone marrow of an individual that is more than 65 years old.

In aspects of the disclosure, the method may include providing an extracellular matrix that is produced by cells capable of producing CCN1/Cyr61. In some aspects, the cells capable of producing CCN1/Cyr61 may include marrow stromal cells, skin cells, muscle cells, or any combination thereof. In some instances, the cells capable of producing CCN1/Cyr61 include cells that are from an individual no more than 65 years old. In some instances, wherein the cells capable of producing CCN1/Cyr61 include cells that contain recombinant DNA fragments for CCN1/Cyr61 expression. In some aspects, the extracellular matrix comprises CCN1/Cyr61 and other extracellular matrix proteins including fibronectin, collagen, RGD-CAP/Betaig-h3, EMILIN-1, periostin, biglycan, thrombospondin-1, tenascin, heparin sulfate, proteoglycan, fibulin-1, galectin-1, decorin, lumican, fibulin-2, ostenectin, fibrillin-2, or any combinations thereof.

In some aspects, the extracellular matrix can be produced by cultivating the cells capable of producing CCN1/Cyr61 in a culture media for 5 to 7 days to form a cell culture mixture containing the extracellular matrix. In some aspects, the produced extracellular matrix can be harvested from cell culture mixture by decellularization using 0.5% Triton X-100 containing 20 mM NH₄OH in PBS. In some aspects, presence of CCN1/Cyr61 in the produced extracellular matrix may be confirmed by reverse transcription polymerase chain reaction (RT-PCR) at RNA levels, and/or immunofluorescence staining and Western Blot analysis at protein levels.

In some aspects, CCN1/Cyr61 may be a cysteine-rich protein coded by a serum-inducible immediate-early gene. In some aspects, gene structure for coding CC1/Cyr61 contains 5 exons and 4 introns with each exon coding for a modular domain with sequence homology to insulin-like growth factor binding proteins (IGFBP), the von Willebrand factor C (VWC) domain, thrombospondin type 1 (TSP-1) domain, and a carboxyl-terminal domain that contains a cysteine knot motif. In some aspects, CC1/Cyr61 may have cell type specific and context sensitive effects.

In aspects of the disclosure, the method may include cultivating the stem cells in need of restoring stem cell properties on the extracellular matrix under culture conditions sufficient to multiply the stem cells and form a rescued stem cell culture that exhibits restored stem cell properties. In some aspects, the culture conditions may include a culture temperature in a of about 37° C. In some aspects, the culture conditions may include an ambient atmospheric carbon dioxide concentration of about 5 vol. %. In some aspects, the culture conditions may include αMEM (minimum essential medium) supplemented with glutamine and 15% fetal bovine serum. In some aspects, the culture conditions may include cultivating the stem cells on tissue culture plastic (TCP).

In some aspects, the rescued stem cell culture having restored stem cell properties (e.g., improved stem cell quality) is from the same donor as the stem cells in need of restoring stem cell properties. Therefore, in some aspects, the rescued stem cell culture may be suitable for autologous cell based-therapies. In some instances, the autologous stem cell based therapies may be adapted to treat conditions including osteoarthritis, general injury, graft versus host disease, lupus, multiple sclerosis, rheumatoid arthritis, Type I diabetes, or any combinations thereof.

In aspects of the disclosure, the rescued stem cell may be used in a method of treating an aged mesenchymal stem cell-related condition for an individual in need thereof. The method may include administering the rescued stem cell culture, including rescued mesenchymal stem cells, to the individual at a dosage sufficient to alleviate the aged mesenchymal stem cell related condition. In some aspects, the mesenchymal stem cells from the individual in need of the treatment of an aged mesenchymal stem cell-related condition may not be able to produce sufficient CNN1/Cyr61 in the extracellular matrix to restore stem cell properties to autologous mesenchymal stem cells. The administering of the rescued stem cell culture may be adapted to reverse the microenvironment of mesenchymal stem cells of the individual, delay progression of aging-related diseases for the individual, delay aging process of the individual, or any combination thereof. In some instances, the aging-related disease may include cardiovascular disease cerebrovascular disease, high blood pressure, cancer, type 2 diabetes, Parkinson's disease, Alzheimer's disease, chronic obstructive pulmonary disease, osteoarthritis, osteoporosis, age-related macular degeneration, hearing loss, or any combination thereof.

In aspects of the disclosure, the stem cells in need of restoring stem cell properties and the cells capable of producing CCN1/Cry6 may be from any mammal. The rescued stem cell culture may be used for autologous stem cell based therapy for the mammal. In some instances, the mammal may include a mouse, the stem cell in need of restoring stem cell properties may include may be mesenchymal stem cells from a mouse older than 18 months. The stem cell capable of producing CCN1/Cry6 may be bone marrow stromal cells from a mouse younger than 3 months.

As part of the disclosure of the present disclosure, specific examples are included below. The examples are for illustrative purposes only and are not intended to limit the disclosure. Those of ordinary skill in the art will readily recognize parameters that can be changed or modified to yield essentially the same results.

### EXAMPLE 1

### Providing Cyr61 Carried in Carbon Nanotubes to Aged Mice

Previously studies showed that extracellular matrices (ECMs), produced ex vivo by various types of stromal cells, direct bone marrow mesenchymal stem cells (BM-MSCs) in a tissue-specific manner and recapitulate physiologic changes characteristic of the aging microenvironment. In particular, BM-MSCs obtained from elderly donors and cultured on ECM produced by young BM stromal cells showed improved quantity, quality and osteogenic differentiation. In aspects herein, the inventors searched for matrix components that are required for a functional BM-MSC niche by comparing ECMs produced by BM stromal cells from “young” (˜25 y/o) versus “elderly” (˜60 y/o) donors. With increasing donor age, ECM fibrillar organization and mechanical integrity deteriorated, along with the ability to promote BM-MSC proliferation and responsiveness to growth factors. Proteomic analyses revealed that the matricellular protein, Cyr61/CCN1, was present in young, but undetectable in elderly, BM-ECM. To assess the role of Cyr61 in the BM-MSC niche, the inventors used genetic methods to down-regulate the incorporation of Cyr61 during production of young ECM and up-regulate its incorporation in elderly ECM. The results showed that Cyr61-depleted young ECM lost the ability to promote BM-MSC proliferation and growth factor responsiveness. However, up-regulating the incorporation of Cyr61 during synthesis of elderly ECM restored its ability to support BM-MSC responsiveness to osteogenic factors such as BMP-2 and IGF-1. The inventors next examined aging bone and compared bone mineral density and Cyr61 content of L4-L5 vertebral bodies in “young” (9-11 m/o) and “elderly” (21-33 m/o) mice. The analyses showed that low bone mineral density was associated with decreased amounts of Cyr61 in osseous tissue of elderly versus young mice. The results strongly demonstrate a novel role for ECM-bound Cyr61 in the BM-MSC niche, where it is responsible for retention of BM-MSC proliferation and growth factor responsiveness, while depletion of Cyr61 from the BM niche contributes to an aging-related dysregulation of BM-MSCs. The results also suggest new potential therapeutic targets for treating age-related bone loss by restoring specific ECM components to the stem cell niche.

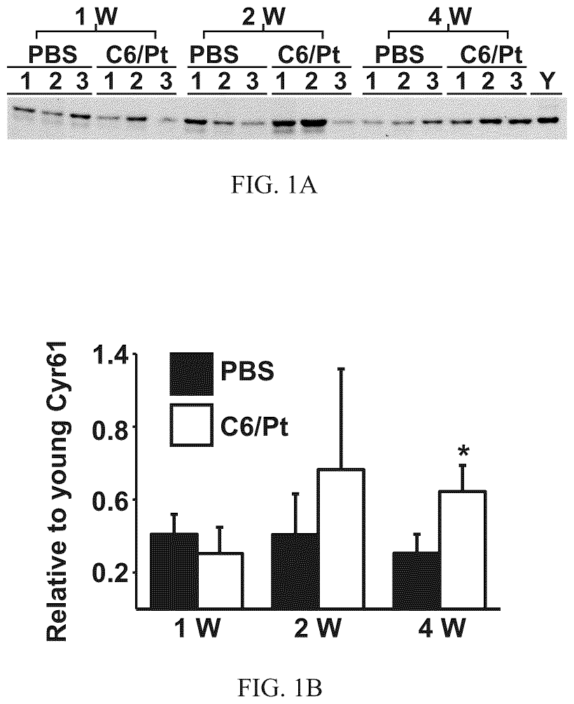

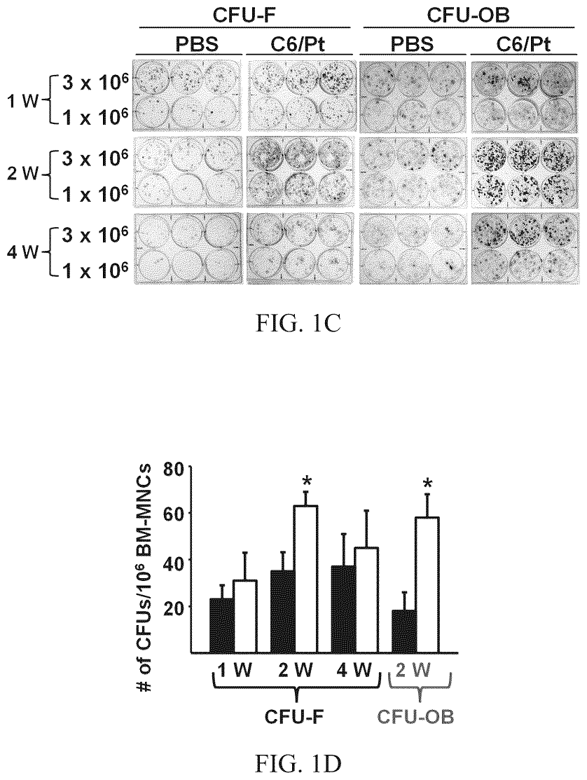

Aged mice (12-18 months old) injected intravenously with recombinant Cyr61 (rCyr61, 50μg/mouse), carried on a bone-targeting vehicle, multi-walled carbon nanotubes (MWCNTs), restored Cyr61 to their bone matrix and attenuated bone loss, along with an increase in colony forming units (CFU) efficiency. This shows the restored Cyr61 to the bone matrix restored increased bone marrow-derived mesenchymal stem cells (BM-MSCs) (FIGS. 1 & 2).

Turning to FIG. 1, eighteen-month-old mice (6 mice/group, 3 male & 3 female) were injected i.v. with a 2:1 ratio of rCyr61/MWCNTs (50 μg rCyr61+25 μg MWCNTs) or PBS and then euthanized after 1, 2 and 4 weeks. Lumbar (L)1-5 vertebrae were dissected and processed for Western blot (Wb) analysis. BM cells were harvested from the femurs and used for colony forming units (CFUs) assay. The results showed that Cyr61 appeared in the bone matrix by 2 weeks after injection and its presence continued to significantly increase through 4 weeks (FIGS. 1A & 1B). Interestingly, CFU-fibroblasts (CFU-F) and CFU-osteoblasts (CFU-OB) reached a peak at 2 weeks post-injection of rCyr61/MWCNTs (FIGS. 1C & 1D).

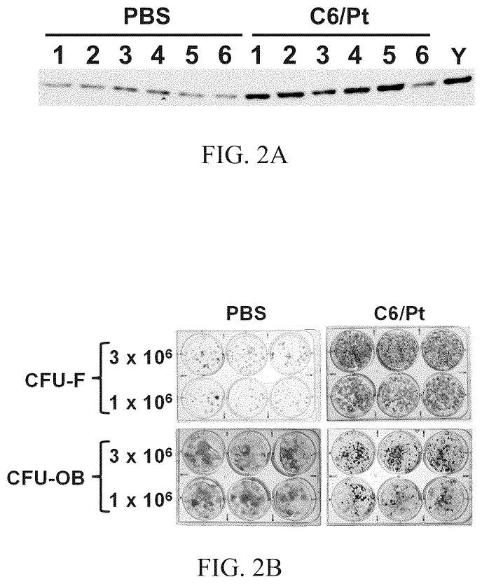

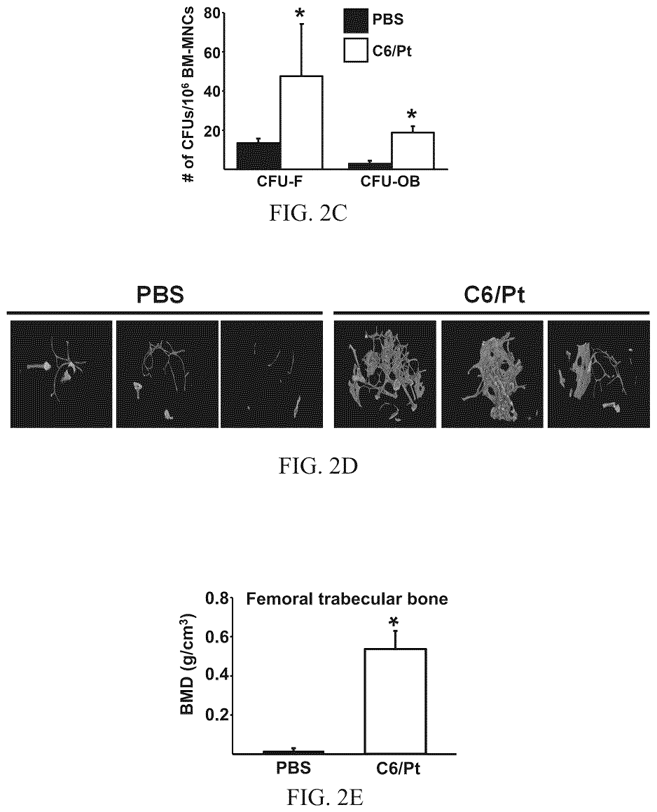

Turning to FIG. 2, Twelve-month-old mice (6 mice/group) were injected i.v. with rCyr61/MWCNTs (2:1 ratio, 50 μg/25 μg) in 200 μl PBS or PBS alone for 3 times at 2-week intervals. At approximately 160 days after the 1^{st} injection, the mice were killed. The L1-3 vertebrae were dissected for Wb analysis and BM cells were harvested from the left femur and both tibias for CFU assay. The right femur and LA-5 vertebrae were used for quantifying bone microstructure using μCT. The results showed that Cyr61 was enriched in the bone matrix of all mice injected with rCyr61/MWCNTs as compared to PBS alone (FIG. 2A). BM cells from mice injected with rCyr61/MWCNTs contained much higher numbers of BM-MSCs, represented by CFU-F and CFU-OB, than mice injected with PBS alone (FIGS. 2B & 2C). Trabecular architecture of the distal femur was imaged, and BMD quantified by μCT; the results showed that mice injected i.v. with rCyr61/MWCNTs had better quantity and quality of femoral trabecular bone than mice injected with PBS alone (FIGS. 2D & 2E).

Overall, it is evident that ECM-bound Cyr61 is a critical component in the BM-MSC niche, where it is responsible for the retention of MSC stemness, while depletion of Cyr61 from the BM-MSC niche contributes to an age-related dysregulation of MSCs, resulting in both a loss of bone mass and bone anabolic response to intermittent PTH treatment. The data provided herein strongly indicate that replenishing Cyr61 to the aging bone matrix will restore the ability of the MSC microenvironment (niche) to support MSC self-renewal, differentiation, and bone formation capacity, resulting in an attenuation of bone loss and improvement in bone anabolic response to intermittent PTH treatment.

The purpose of this study is to better understand the role of Cyr61 in osteogenesis. Cyr61 (CCN1) is a member of the Cell Communication Network protein family which has binding domains for insulin-like growth factor binding protein (IGFBP), TGF-β, and BMPs. In addition, this protein also tightly binds fibronectin in the extracellular matrix (ECM) and interacts with cells via integrins. Bone specific Cyr61 knockout mice exhibit decreased bone mass.

Methods: Twelve month-old male and female C57B6 mice were injected intravenously (i.v.) 3 times (every 2 weeks) with recombinant human (rh) Cyr61 that was carried by a bone-targeting vehicle, multi-walled carbon nanotubes (MWCNTs) (rhCyr61/MWCNTs: 50 μg/25 μg), in 200 μl PBS or PBS alone. Whole-body BMD was measured by dual-energy X-ray absorptiometry (DXA) every month until euthanasia at 160 days following the 1st injection. Lumbar vertebrae (L1-3) were harvested for Western blot (Wb) analysis and BM cells were obtained from the left femur and both tibias for colony forming unit (CFU) assay. Right femur and L4-5 were scanned by μCT to quantify changes in bone structure.

Results: Wb analysis showed that Cyr61 was more abundant in the bone matrix of mice treated with rhCyr61/MWCNTs compared to PBS alone. Furthermore, BM cells from the rhCyr61/MWCNT-treated group had 2-3 times more CFU-fibroblasts and CFU-osteoblasts than those treated with PBS alone. Mice treated with rhCyr61/MWCNTs also exhibited a significant increase in femoral trabecular bone compared to mice treated with PBS alone. Live measurements of whole-body BMD revealed that aged mice injected with rhCyr61/MWCNTs experienced an ˜20% increase in BMD, a benefit not observed in mice injected with PBS alone.

### EXAMPLE 2

### Rejuvenating the Aged Skeletal Microenvironment: Reversing Bone Loss by Replenishing Cyr61/CCN1, a Key Stem Cell Niche Component Depleted with Aging

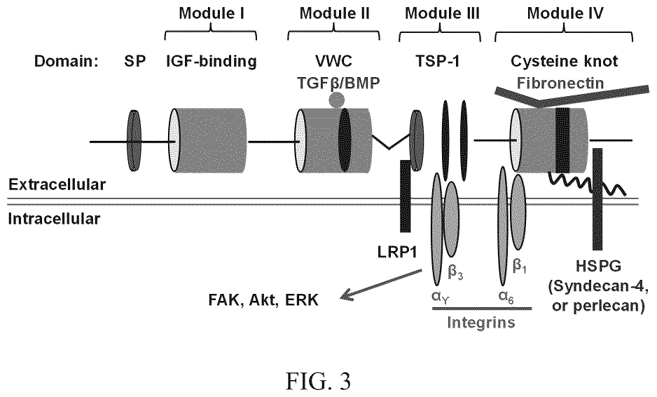

Cyr61 (CCN1) is a member of the Cell Communication Network protein family which has binding domains for insulin-like growth factor binding protein (IGFBP), TGF-β, and BMPs (FIG. 3). Previously, we showed that Cyr61 levels in the bone marrow (BM) mesenchymal stem cell (MSC) niche decreased with age (Marinkovic M et al. Matrix-bound Cyr61/CCN1 is required to retain the properties of the bone marrow mesenchymal stem cell niche but is depleted with aging. Matrix Biol. 2022; 111:108-32.). This depletion impairs MSC self-renewal, differentiation, and BMP responsiveness, leading to bone loss (Marinkovic M et al.). In the present study, we hypothesized that restoration of this protein to the aged bone matrix promoted MSC self-renewal, differentiation, and bone formation capacity, resulting in attenuated bone loss.

Male and female C57BL/6 mice (12-18 months old) were injected intravenously (i.v.) 3 times (every 2 weeks) with recombinant Cyr61 (rCyr61) that was carried by a bone-targeting vehicle, multi-walled carbon nanotubes (MWCNTs) (rCyr61/MWCNTs: 50 μg/25 μg), in 200 μl PBS or PBS alone. Whole-body BMD was measured by dual-energy X-ray absorptiometry (DXA) every month until euthanasia on day 160 following the 1st injection. Lumbar vertebrae (L1-3) were harvested for Western blot (Wb) analysis to validate the presence of the injected rhCyr61. BM cells were obtained from the left femur and both tibias for colony forming unit (CFU) assay. Right femur and L4-5 were scanned by μCT to quantify changes in bone structure.

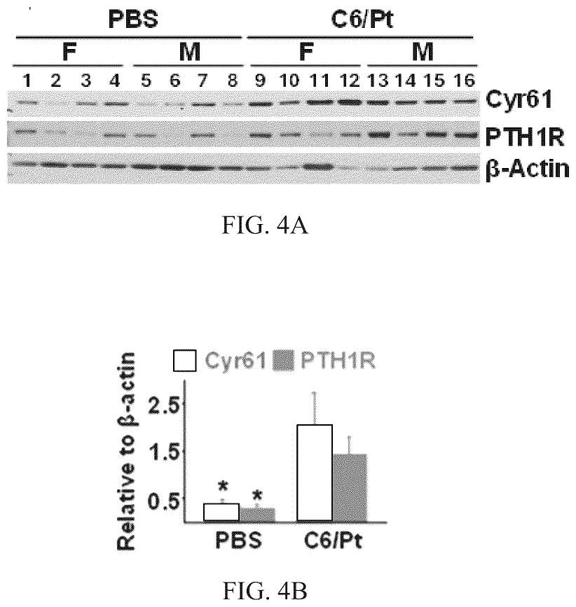

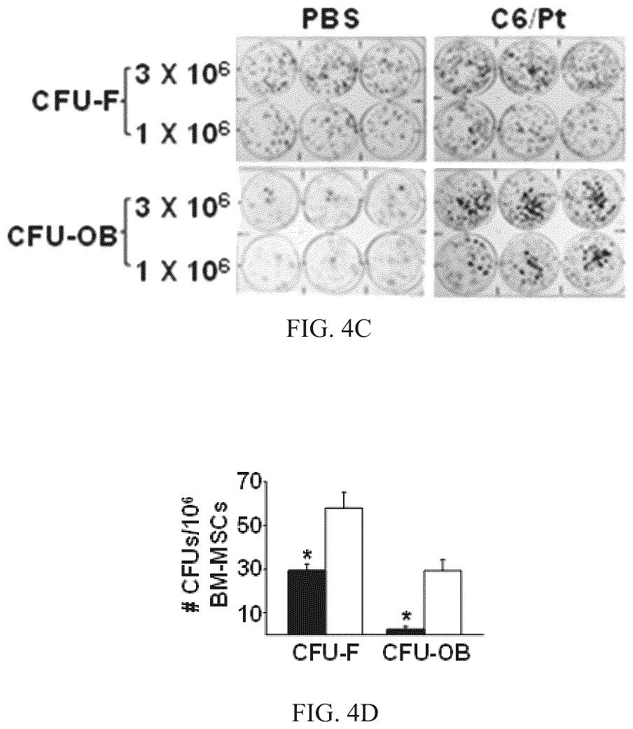

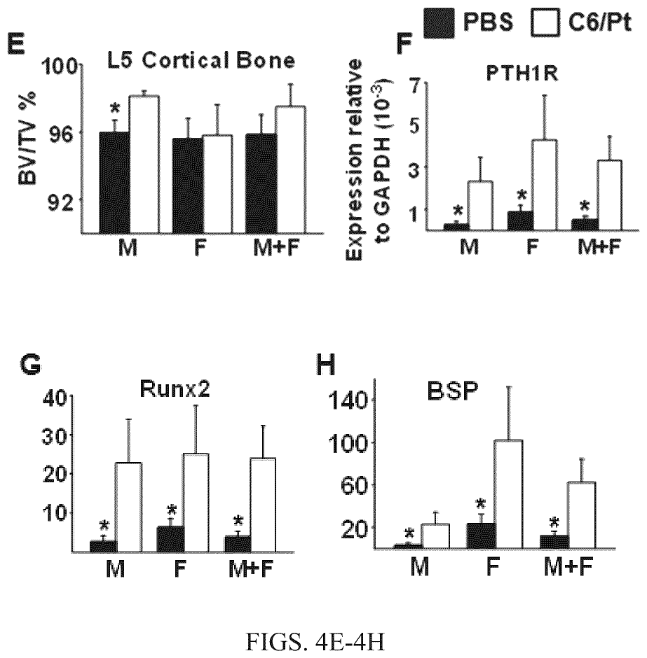

As shown in FIG. 4, Old mice treated with rCyr61/MWCNTs rapidly achieved an increase in CFU number, parathyroid hormone receptor (PTH1R) expression, and osteogenesis. 18-month-old male (M) or female (F) mice were injected (i.v.) 3 times at 2-week intervals with rCyr61/MWCNTs (C6/Pt) in 200 μl PBS or PBS alone. At 4 weeks after the last injection, mice were euthanized for the various assays.

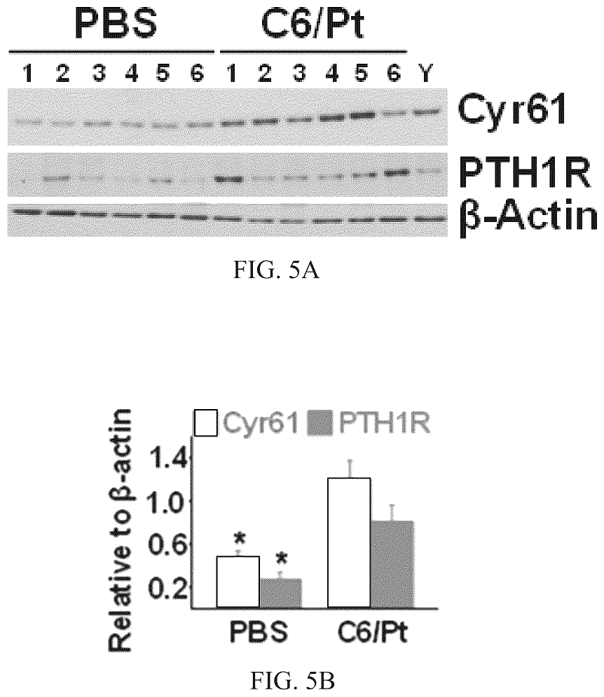

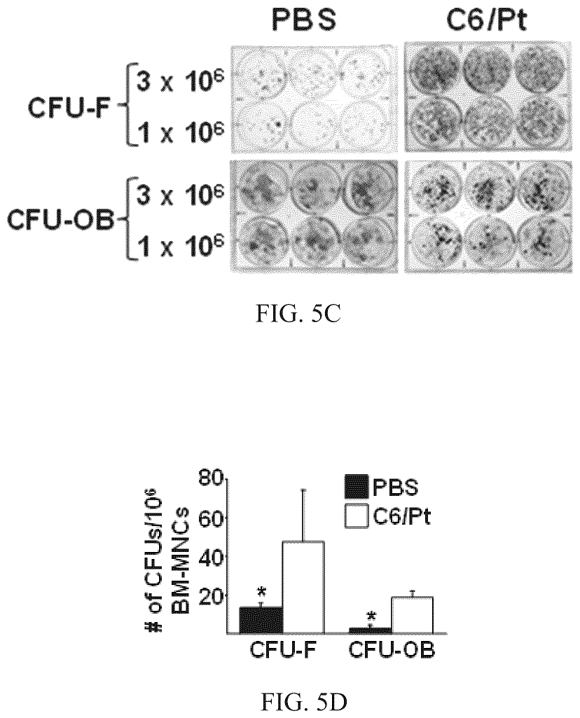

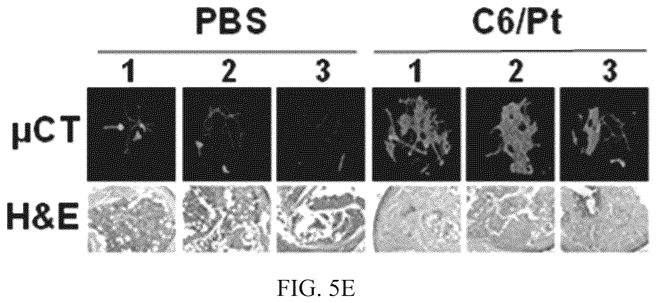

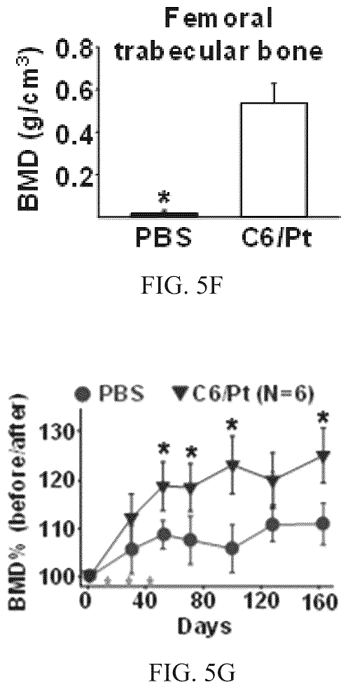

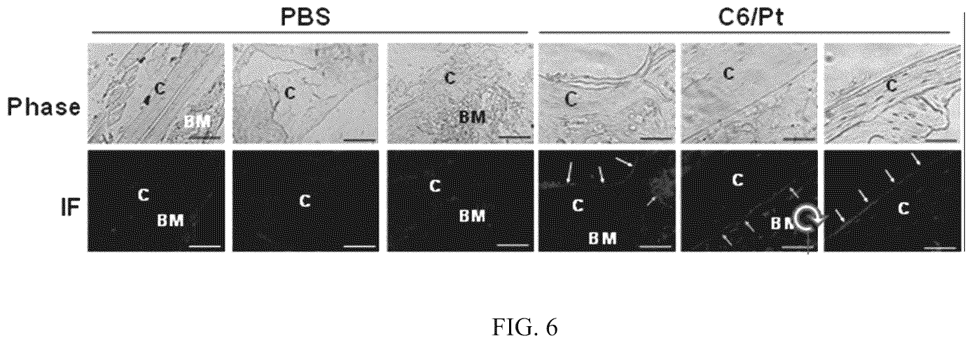

As shown in FIG. 5, Mice treated with rCyr61/MWCNTs display long term effects on increased CFUs in BM and bone mass. 12-month-old male (M) or female (F) mice were injected (i.v.) 3 times at 2-week intervals with rCyr61/MWCNTs (C6/Pt) in 200 μl PBS or PBS alone. At day 160 after the 1st injection, mice were euthanized for the various assays.

As shown in FIG. 6, Cyr61 is mainly localized to the periosteum (lining cells) and endosteal BM sinuses

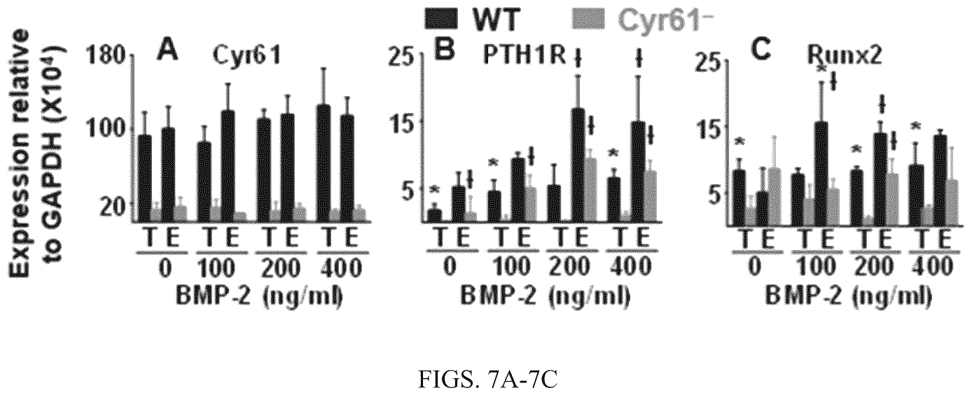

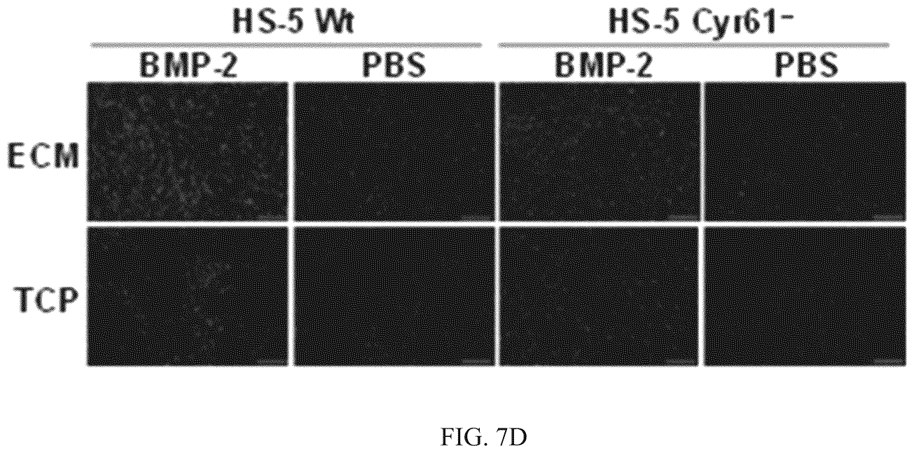

As shown in FIG. 7, Cyr61 knock out (Cyr61−) HS-5 cells failed to express PTHIR, but expression was significantly rescued by culture on human (h) BM-ECM. Cyr61 in HS-5 cells [a human BM stromal cell line (Adamo A et al. HS-5 and HS-27A stromal cell lines to study bone marrow mesenchymal stromal cell-mediated support to cancer development. Front Cell Dev Biol. 2020; 8:584232.)] was knocked out (Cyr61−) using CRISPR technology. Cyr61− or wild type (Wt) HS-5 cells were cultured on tissue culture plastic (T) or young hBM-ECM (E), which naturally contains ECM-bound Cyr61 (1). After 7 days in culture, the cells were treated with varying concentrations (0 to 400 ng/ml) of BMP-2 for 48 hours.

The findings clearly indicate that ECM-bound Cyr61 is critical for maintaining BM-MSC function, PTHIR expression, and osteoblast differentiation. As shown, aged skeletal microenvironment is rescued by replenishing a key ECM component, CCN1/Cyr61, which is essential for retaining the self-renewal and differentiation capacity of BM-MSCs (Marinkovic M et al.). The aspects herein show a new therapeutic approach that has the potential to not only treat or retard age-related bone loss, but also synergistically improve current treatments, such as intermittent PTH, while minimizing side effects. Moreover, the present study establishes a foundation where this approach can be used to explore restoration of other important ECM proteins, known to play a critical role in osteogenesis (e.g., biglyan & decorin, small leucine-rich proteoglycans), as potential therapies.

Although aspects of the present application and their advantages have been described in detail, it should be understood that various changes, substitutions and alterations can be made herein without departing from the spirit and scope of the aspects as defined by the appended claims. Moreover, the scope of the present application is not intended to be limited to the particular aspects of the process, treatment, machine, manufacture, composition of matter, means, methods, and/or steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding aspects described herein may be utilized. Accordingly, the appended claims are intended to include within their scope such processes, machines, manufacture, compositions of matter, means, methods, or steps.

1. A composition comprising recombinant Cyr61 carried in carbon nanotubes.

2. The composition of claim 1, wherein the carbon nanotubes comprise multi-walled carbon nanotubes.

3. The composition of claim 1, wherein the carbon nanotubes are bone-targeting carbon nanotubes.

4. The composition of claim 1, wherein the recombinant Cyr61 and carbon nanotubes are at approximately a 2:1 ratio.

5. The composition of claim 1, wherein the composition is formulated for intravenous administration.

6. The composition of claim 1, wherein the recombinant Cyr61 is at a concentration of approximately 2 milligram per gram of composition.

7. A method of targeting Cyr61 to bone tissue in a patient, the method comprising administering comprising recombinant Cyr61 carried in carbon nanotubes to the patient.

8. The method of claim 7, wherein the patient has been found to have reduced Cyr61, and/or parathyroid hormone (PTH) receptor compared to a control.

9. The method of claim 8, wherein the control is a measured amount of Cyr61, PTH, and/or PTH receptor in a healthy individual.

10. (canceled)

11. The method of claim 8, wherein the control is a baseline amount of Cyr61, PTH, and/or PTH receptor measured in the patient.

12. The method of claim 7, wherein the patient has received, or is indicated to receive, an intermittent PTH treatment.

13. (canceled)

14. The method of claim 7, any wherein the composition is administered intravenously.

15. The method of claim 7, further comprising administering PTH and/or a PTH analog to the patient.

16.-26. (canceled)

27. A method for treating a patient having, suspected of having, diagnosed with having, or having symptoms of osteopenia or osteoporosis, the method comprising administering a composition comprising recombinant Cyr61 carried in carbon nanotubes to the patient.

28. The method of claim 27, wherein the patient has been found to have reduced Cyr61, parathyroid hormone (PTH), and/or PTH receptor compared to a control.

29. The method of claim 28, wherein the control is a measured amount of Cyr61, PTH, and/or PTH receptor in a healthy individual.

30. (canceled)

31. The method of claim 28, wherein the control is a baseline amount of Cyr61, PTH, and/or PTH receptor measured in the patient.

32. The method of claim 27, wherein the patient has been found to have reduced Cyr61, parathyroid hormone (PTH), and/or PTH receptor in a bone tissue sample from the patient.

33. (canceled)

34. The method of claim 27, wherein the composition is administered intravenously.

35. The method of claim 27, further comprising administering PTH and/or a PTH analog to the patient.

36. (canceled)
